(19) United States
(12) Plant Patent Application Publication
(10) Pub. No.: US 2014/0223619 P1
(43) Pub. Date: Aug. 7, 2014

(54) Blueberry plant named 'C03-038'

(50) ;

(71) Applicants: Florida Foundation Seed Producers, Inc., Marianna, FL (US); CostaExchange Pty Ltd., Corindi (AU)

(72) Inventors: Gary Wright, Corindi Beach (AU); Paul Lyrene, Micanopy, FL (US)

(21) Appl. No.: 13/999,294

(22) Filed: Feb. 6, 2014

Related U.S. Application Data
(60) Provisional application No. 61/849,978, filed on Feb. 6, 2013.

(51) Int. Cl. A01H 5/00

(52) U.S. Cl. PLT/157

(57) ABSTRACT

A new and distinct cultivar of blueberry (Vaccinium corymbosum hybrid) plant named ‘C03-038,’ characterized by its semi-upright plant shape and strong plant growth vigor, low chilling requirement, early timing of fruiting, medium fruit size with oblate shape, firm fruit suited to handling, good fruit flavor. This combination results in higher quality fruit with a later availability than other varieties.

### LATIN NAME OF THE FAMILY, GENUS, AND SPECIES

Family—Ericaceae

Genus—Vaccinium

Species—corymbosum hybrid

### VARIETY DENOMINATION

The new blueberry plant claimed is of the variety denominated ‘C03-038.’

### BACKGROUND OF THE INVENTION

The present invention relates to a new and distinct perennial variety of Vaccinium corymbosum hybrid (blueberry), which has been given the variety denomination of ‘C03-038.’ The new variety ‘C03-038’ shows distinctive traits such as early season, good flavor and firm fruit. Its market class is that of a fruiting plant. The new variety ‘C03-038’ is intended for use as fresh fruit for shipping, customer pick and processing markets and as a home garden plant.

The new blueberry cultivar is a selection resulting from seedlings produced in a controlled breeding programme of Vaccinium varieties in Florida, USA in 2001 from a cross of the blueberry variety known as ‘F93-368’ (seed parent) (unpatented) and the blueberry variety known as ‘C97-390’ (pollen parent) (U.S. Plant Pat. No. 20,642). The seed from the cross was sown and grown in Corindi Beach, New South Wales, Australia. The new cultivar was discovered and selected in 2003 as a single plant within a population of seedlings resulted from the controlled cross, in an experimental block in the field at Corindi Beach, New South Wales, Australia, and has since been named ‘C03-038.’ Selection criteria were a combination of early season, low chilling requirement, medium fruit of good flavor, firm fruit. The new variety was subsequently evaluated for a number of years at the commercial farm at Corindi Beach, New South Wales, Australia.

Asexual reproduction of the new variety ‘C03-038’ by softwood cutting propagation since 2003 at Corindi Beach, New South Wales, Australia has demonstrated that the new variety reproduces true to type with all of the characteristics, as herein described, firmly fixed and retained through successive generations of such asexual propagation, with the clones phenotypically identical to the original plant.

The seed parent ‘F93-368’ is characterized by a medium fruit size and very early to early timing of ripening of fruit. The pollen parent ‘C97-390’ is characterized by a large fruit size and very early to early timing of ripening of fruit. The new variety ‘C03-038’ differs from the seed and pollen parents in that ‘C03-038’ has a later cropping timing compared to both parents and a smaller fruit size compared to the pollen parent. The new variety ‘C03-038’ has maintained its distinguished characteristics throughout successive asexual propagation.

### SUMMARY OF THE INVENTION

The following characteristics of the new variety have been repeatedly observed and can be used to distinguish ‘C03-038’ as a new and distinct variety of Vaccinium corymbosum hybrid:
1. low chilling requirement
2. semi-upright habit
3. early fruiting season
4. medium fruit size
5. good flavor
6. firm fruit

Plants of the new variety ‘C03-038’ differ from plants of the parents primarily in a later cropping timing compared to both parents and a smaller fruit size compared to the pollen parent. The new blueberry variety ‘C03-038’ has maintained its distinguished characteristics throughout successive asexual propagation.

### BRIEF DESCRIPTION OF THE DRAWINGS

The accompanying photographic illustration shows typical specimens in full color of the foliage and fruit of the new variety ‘C03-038.’ The colors are as nearly true as is reasonably possible in a color representation of this type.

FIG. 1 is a photograph of the new variety ‘C03-038’ demonstrating the plant's semi-upright growth habit.

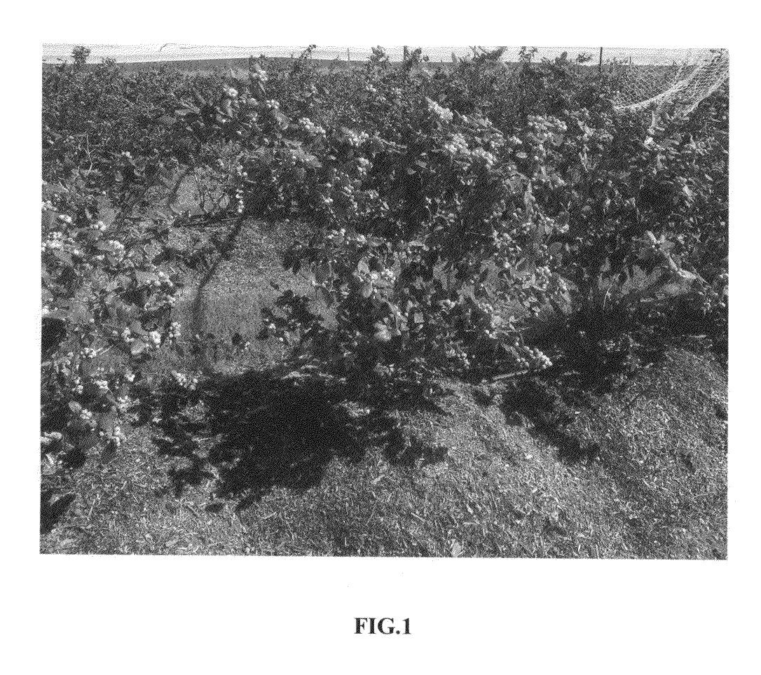

FIG. 2 is a photograph of the fruit of the new variety ‘C03-038.’

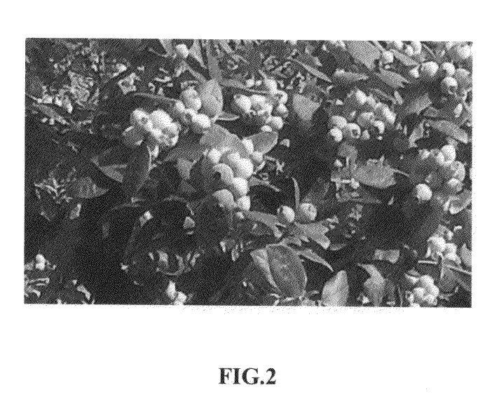

FIG. 3 is a photograph of the green fruit of the new variety ‘C03-038.’

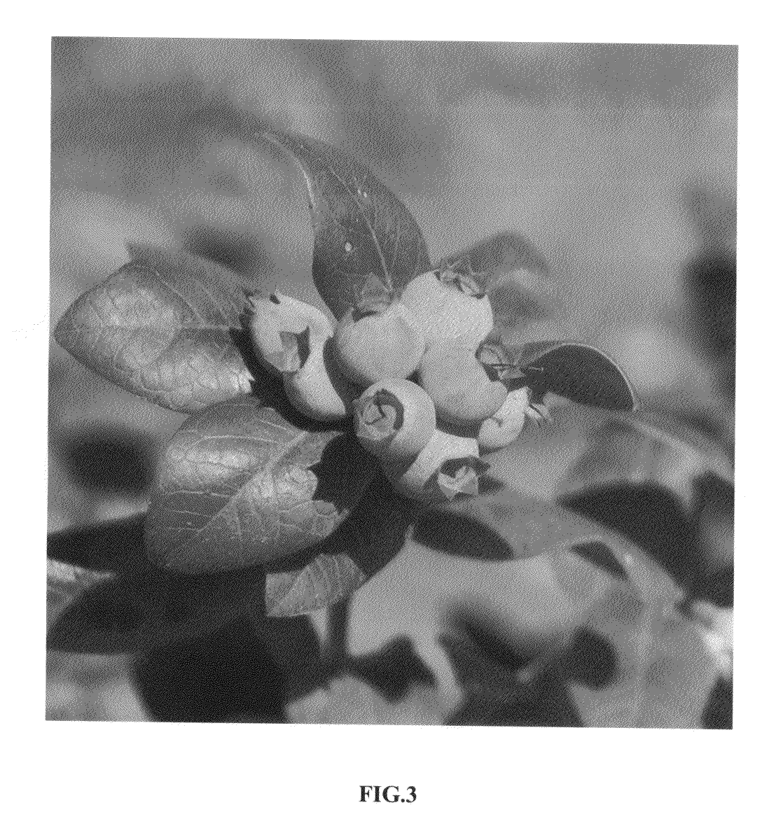

FIG. 4 is a photograph of the flowers of the new variety ‘C03-038.’

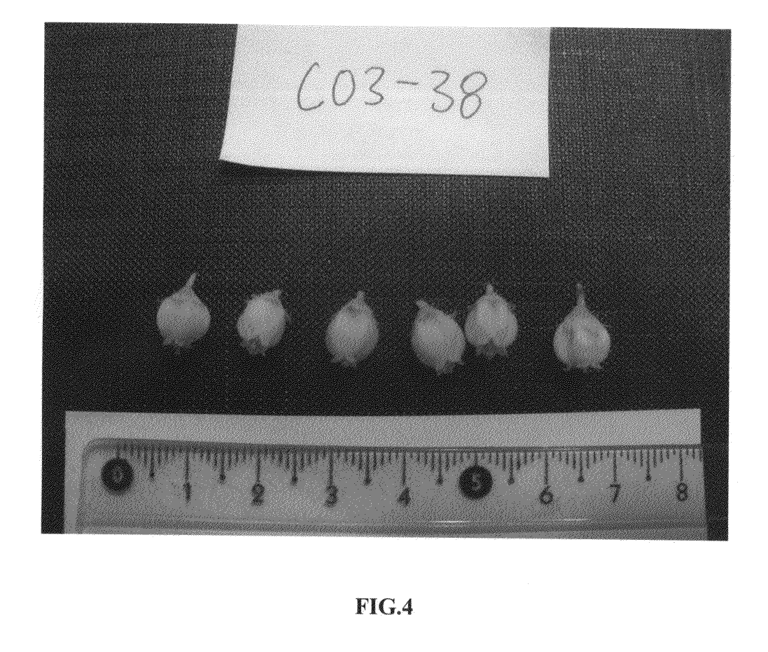

FIG. 5 is a photograph of the new variety ‘C03-038’ growing in commercial field.

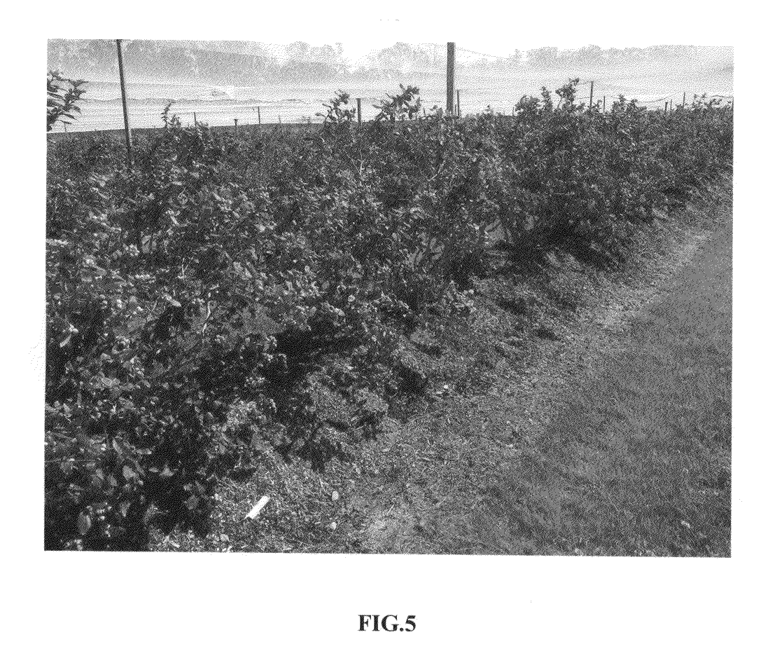

The colors in the photographs are as close as possible with the photographic and printing technology utilized. The color values cited in the detailed botanical description accurately describe the colors of the new blueberry.

### DETAILED BOTANICAL DESCRIPTION

The following detailed description sets forth the distinctive characteristics of ‘C03-038.’ The data which defines these characteristics was collected from asexual reproductions of the original selection. Dimensions, sizes, colors, and other characteristics are approximations and averages set forth as accurately as possible. The plant history was taken on plants approximately 5 years of age, and the descriptions relate to plants grown in the field in Corindi Beach, New South Wales, Australia. Descriptions of fruit characteristics were made on fruit grown in Corindi Beach, New South Wales, Australia. Color designations are from R.H.S. Chart—edition 2007.
Classification:
   Family.—Ericaceae.
   Genus.—Vaccinium.
   Species.—corymbosum hybrid.
   Common name.—blueberry.
Parentage:
   See parent.—“F93-368’ (unpatented).
   Pollen parent.—‘C97-390’ (U.S. Plant Pat. No. 20,642).
Market class: Suitable for the Commercial, Hand Harvest Fresh, Processed, and Home Garden Markets
Plant:
   Growth habit.—Semi-upright.
   Growth.—Vigorous.
   Productivity.—High.
   Chilling requirement.—Low chill.
   Tolerance to disease.—No major diseases were found to affect the new variety.
   Leafing.—Medium to high; Retains leaves overwinter under the environment at Corindi Beach, New South Wales, Australia.
Stem:
   Suckering tendency.—Medium.
Foliage:
   Time of beginning of leaf bud burst.—The end of August with average date at August 28^{th}; Similar to that of ‘C97-390.’
   Leaf color (top side).—Similar to green group 137B and the vein color similar to yellow-green group 145A.
   Leaf shape.—Elliptic.
   Leaf margins.—Entire.
   Leaf length.—Mean 49.75 mm (45-58 mm range).
   Leaf width.—Mean 28.70 mm (24-36 mm range).
   Leaf nectarines.—Absent.
   Pubescence of upper side.—Absent.
   Petioles.—a. Length: Mean 2.32 mm (2-4 range).
Flowers:
   Time of 50% anthesis.—Average date at August 10^{th}.
   Flower shape.—Campanulate.
   Flower fragrance.—None.
   Corolla.—a. Color: White. b. Width: Mean 4.47 mm (3-5 mm range) c. Anthocyanin Coloration of Corolla: Absent d. Corolla Ridges: Present.
Fruit:
   Time of fruit ripening.—Early season.
   Time of 50% maturity.—Average date October 1^{st}.
   Berry weight.—Mean 2 g (1.7-2.5 range).
   Berry shape.—Oblate.
   Uses.—Hand harvest fruit.

1. A new and distinct variety of blueberry plant named ‘C03-038,’ substantially as illustrated and described herein.
